(19) United States
(12) Patent Application Publication
(10) Pub. No.: US 2004/0131619 A1
(43) Pub. Date: Jul. 8, 2004

(54) Thymic atrophy

(75) Inventors: Gregory D. Sempowski, Durham, NC (US); Barton F. Haynes, Durham, NC (US)

Correspondence Address: NIXON & VANDERHYE, PC, 1100 N GLEBE ROAD, 8TH FLOOR, ARLINGTON, VA, 22201-4714, US

(73) Assignee: DUKE UNIVERSITY, Duham, NC

(21) Appl. No.: 10/673,667

(22) Filed: Sep. 30, 2003

Related U.S. Application Data
(60) Provisional application No. 60/414,366, filed on Sep. 30, 2002.

(51) Int. Cl. A61K 39/395; A61K 38/17

(52) U.S. Cl. 424/155.1; 514/12

(57) ABSTRACT

The present invention relates, in general, to thymic atrophy and, in particular, to a method of treating or preventing thymic atrophy in septic shock.

This application claims priority from U.S. Provisional Application No. 60/414,366, filed Sep. 30, 2002, the entire content of which is incorporated herein by reference.

### TECHNICAL FIELD

The present invention relates, in general, to thymic atrophy and, in particular, to a method of treating or preventing thymic atrophy in septic shock.

### BACKGROUND

Septic shock is the most common cause of death in critical care units in the United States (Stone, Science 264:365 (1994), Billiau and Vandekerckhove, European Journal of Clinical Investigation 21:559 (1991)). Data are emerging that morbidity and mortality from septic shock may be directly related to sepsis-induced apoptosis of lymphocytes, in spleen, lymph node, and thymus (Hotchkiss et al, Critical Care Medicine 27:1230 (1999), Wang et al, Journal of Immunology 152:5014 (1994), Oberholzer et al, FASEB Journal 15:879 (2001)). Oberholzer et al. have recently shown that intrathymic injection of adenovirus expressing IL-10 prevented thymocyte apoptosis and thymic atrophy, and prevented sepsis-induced death in mice (Oberholzer et al, FASEB Journal 15:879 (2001), Oberholzer et al, PNAS 89:11503 (2001)). While TNF-α and IL-1 have been implicated in apoptosis and organ dysfunction in sepsis, treatment of patients in septic shock with TNF-α or IL-1 antagonists did not improve survival (Abraham et al, Lancet 351:929 (1998), Fisher et al, JAMA 271:1836 (1994)). These data suggested that cytokines in addition to TNF-α and IL-1 may play critical roles in mediation of apoptosis and mortality in sepsis.

Leukemia inhibitory factor (LIF) is a member of the IL-6 cytokine family (LIF, OSM, CNTF and IL-6) and stimulates proliferation of hematopoietic progenitors, maintains the developmental potential of embryonic stem cells by suppressing differentiation, and promotes differentiation of cholinergic neurons (Metcalf, Growth Factors 7:169 (1992), see also Kurzrock et al, Endocrine Reviews 12:208-217 (1991)). LIF is expressed by a variety of cell types, including bone-marrow stromal cells, thymic epithelial cells, fibroblasts, T lymphocytes and a number of malignancies (Patterson, Current Opinion in Neurobiology 2:94 (1992), Gearing et al, EMBO Journal 6:3995 (1987)).

LIF mRNA levels have been shown to be increased in atrophic human thymus, and injection of LIF IP into BALB/c mice has been shown to induce acute thymic atrophy (Sempowski et al, Journal of Immunology 164:2180 (2000)). Melmed et al. have demonstrated that LIF plays a role in synergizing with corticotropin-releasing hormone (CRH) to regulate pituitary ACTH and adrenal corticosteroid production (Wang et al, Journal of Immunology 152:5014 (1994), Shimon et al, Journal of Clinical Investigation 100:357 (1997), Wang et al, Endocrinology 137:2947 (1996)).

The present invention derives, at least in part, from studies designed to investigate the role of systemic and intrathymic LIF and corticosteroid production in mediating endotoxin-induced acute thymic atrophy. The results obtained demonstrate a key pathway for gram-negative endotoxin induced thymic atrophy wherein LPS induces systemic and intrathymic cortoicosteriod production—a direct mediator of thymocyte apoptosis.

### SUMMARY OF THE INVENTION

The present invention relates generally to thymic atrophy. Specifically, the invention relates to a method of treating or preventing thymic atrophy in septic shock.

Objects and advantages of the present invention will be clear from the description that follows.

### BRIEF DESCRIPTION OF THE DRAWINGS

FIGS. 1A-1D. LPS upregulated serum LIF protein and thymic LIF mRNA. BALB/c mice were injected IP with 100 μg LPS (n=5) or saline (n=6). (FIG. 1A) Serum LIF levels at 1 and 24 hours. (FIG. 1B) LIF mRNA expression (% GAPDH) at 0, 6, 12 and 24 hours (n=3). LPS-induced acute thymus atrophy was inhibited by goat anti-LIF polyclonal antibody. Six BALB/c mice per group were injected IP with either goat IgG (1 mg) or anti-LIF polyclonal goat IgG (1 mg) 6 hours prior to IP injection with either saline or LPS (100 μg). (FIG. 1C) Thymus weight. (FIG. 1D) Thymocyte number. Mean±SEM. * P<0.05.

FIG. 2. LIF-induced acute thymus atrophy was inhibited by metyrapone. Three BALB/c mice per group were injected IP with either saline or metyrapone (30 mg/kg) 24 hours prior to injection with either saline or LIF (2 μg 3× a day IP) for 3 days. Animals were sacrificed and degree of thymus atrophy determined. Mean±SEM. * P<0.05.

FIGS. 3A and 3B. LIF-induced acute thymus atrophy was inhibited by adrenalectomy. Normal and adrenalectomized BALB/c mice (n=3) were injected with either saline or LIF (2 μg 3× a day IP) for 3 days. Animals were sacrificed and degree of thymus atrophy dtermined. (FIG. 3A) Thymus weight. (FIG. 3B) Number of DP thymocytes. Mean±SEM. * P<0.05.

FIG. 4. LIF induced a CS-dependent decrease in CD3⁺ DP thymocytes per thymus in murine FTOC. FTOC were incubated for 3 days with recombinant LIF (10 ng/ml) with and without metyrapone (180 g/ml). Harvested thymocytes were counted and phenotyped. Data are mean±SEM of 9 experiments. * P<0.05 versus medium only.

FIGS. 5A-5D. Acute thymic atrophy and thymic rebound following LPS (100 μg IP) or saline injection. (FIG. 5A) Thymus weight. (FIG. 5B) Absolute number of thymocytes. (FIG. 5C) Absolute number of CD4/CD8 DP thymocytes. (FIG. 5D) Molecules of mTREC per mg thymus tissue. Data are mean±SEM of 3 mice per group. *p<0.05 compared to saline treated.

### DETAILED DESCRIPTION OF THE INVENTION

The present invention relates to a method of treating or preventing gram-negative endotoxin-induced thymic atrophy in a patient. The method comprises administering to a patient in need of such treatment or prevention an agent that inhibits LIF induction of thymic corticosteriods. The invention also relates to methods of screening test compounds for their ability to inhibit LIF induction of thymic corticosteriods and resulting thymic atrophy.

Suitable for use in the invention are agents that inhibit intrathymic production and/or function of LIF (that is, LIF antagonists). Examples of LIF antagonists include antibodies, proteins, peptides, glycoproteins, glycopeptides, glycolipids, polysaccharides, oligosaccharides, nucleic acids, bioorganic molecules, peptidomimetics, transcriptional and translation control sequences, and the like. Another class of antagonists blocks or prevents intracellular or membrane associated events occurring between LIF and its receptor.

In one embodiment, the antagonist is an antibody that binds to LIF and prevents its interaction with its receptor. Suitable antibodies include polyclonal antibodies, monoclonal antibodies (mAbs), humanized or chimeric antibodies, single chain antibodies, Fab fragments, F(ab′)₂ fragments, Fv fragments, fragments produced by a Fab expression library, anti-idiotypic (anti-Id) antibodies, and epitope-binding fragments of any of the above. Any of such antibodies or fragments thereof can be produced by standard immunological methods or by recombinant expression of nucleic acid molecules encoding the antibody or fragment thereof in an appropriate host organism. (See, for example, Kohler and Milstein, Nature 256:495-497 (1975); U.S. Pat. No. 4,376,110;Kosbor et al, Immunology Today 4:72 (1983); Cole et al, Proc. Natl. Acad. Sci. USA 80:2026-2030 (1983); Cole et al, Monoclonal Antibodies and Cancer Therapy, Alan R. Liss, Inc., pp. 77-96 (1985); Morrison et al, Proc. Natl. Acad. Sci. 81:6851-6855 (1984); Neuberger et al, Nature 312:604-608 (1984); Takeda et al, Nature 314:452-454 (1985); U.S. Pat. No. 4,946,778; Bird, Science 242:423-426 (1988); Huston et al, Proc. Natl. Acad. Sci. USA 85:5879-5883 (1988); Ward et al, Nature 334:544-546 (1989); Skerra et al, Science 242:103825-1041 (1988); Huse et al, Science, 246:1275-1281 (1989)). The invention also includes the use of antibodies that antagonistically bind the LIF receptor and inhibit binding of the cytokine thereto.

In another embodiment, the antagonist is a soluble LIF receptor that prevents interaction of the receptor with LIF (for the primary structure of the receptor, see IP et al, Cell 69:1121 (1992)). Suitable soluble receptors can be prepared, for example, by identifying the extracellular domain of the receptor and excising the transmembrane domain therefrom (or otherwise producing (e.g., recombinantly) the extracellular domain).

In yet another embodiment, the antagonist is a bioorganic molecule, for example, an orally active compound, that is based on molecular modeling studies and that is capable of preventing the interaction between LIF and its receptor. Suitable such molecules can, for example, mimic the soluble receptor.

Antisense and ribozyme molecules that inhibit LIF expression can also be used as antagonists in accordance with the invention. Techniques for the production and use of such molecules are well known to those of skill in the art. (For a review of antisense, see Stein, in Ch. 69, Section 5 “Cancer: Principle and Practice of Oncology”, 4th ed., ed. by DeVita et al., J. B. Lippincott, Philadelphia 1993).

An antisense LIF nucleic acid as used herein refers to a nucleic acid capable of hybridizing to a portion of a LIF RNA (preferably mRNA) by virtue of sequence complementarity. The antisense molecules bind to the complementary LIF mRNA transcripts and reduce or prevent translation. The antisense nucleic acid of the invention can be complementary to a coding and/or noncoding region of a LIF mRNA. Absolute complementarity, although preferred, is not required. A sequence having sufficient complementarity to hybridize with the RNA, forming a stable duplex, is all that is necessary. Generally, the longer the hybridizing nucleic acid, the more base mismatches with the RNA it can contain and still form a stable duplex (or triplex, as the case may be). One skilled in the art can ascertain a tolerable degree of mismatch by use of standard procedures to determine the melting point of the hybridized complex.

Nucleic acid molecules that are complementary to the 5′ end of the LIF message, e.g., the 5′ untranslated sequence up to and including the AUG initiation codon, can be used, as can sequences complementary to the 3′ untranslated sequences of the LIF mRNA. (See generally, Wagner, R, Nature 372:333-335 (1994)).

Whether designed to hybridize to the 5′-, 3′- or coding region of target mRNA, antisense nucleic acids are, advantageously, at least six nucleotides in length, and preferably range from 6 to about 50 nucleotides in length.

The antisense molecule can be DNA or RNA or chimeric mixtures or derivatives or modified versions thereof, single-stranded or double-stranded. The antisense molecule can be modified at the base moiety, sugar moiety, or phosphate backbone, for example, to improve stability of the molecule, hybridization, etc. The antisense molecule can include other appended groups such as peptides (e.g., for targeting host cell receptors in vivo), or agents facilitating transport across the cell membrane (see, e.g., Letsinger et al, Proc. Natl. Acad. Sci. U.S.A. 86:6553-6556 (1989); Lemaitre et al, Proc. Natl. Acad. Sci. 84:648-652 (1987); PCT Publication No. WO88/09810, published Dec. 15, 1988). The antisense molecule can be conjugated to another molecule, e.g., a peptide, triggered cross-linking agent, transport agent, hybridization-triggered cleavage agent, etc.

Antisense molecules of the invention can be synthesized by standard methods known in the art, e.g., by use of an automated DNA synthesizer.

Pharmaceutical compositions of the invention comprising an effective amount of a LIF antisense nucleic acid in a pharmaceutically acceptable carrier, can be administered to a patient. The amount of LIF antisense nucleic acid that will be effective can vary with the patient and the effect sought, and can be determined by standard clinical techniques.

A number of methods have been developed for delivering antisense DNA or RNA to cells; e.g., antisense molecules can be injected directly into the tissue site, or modified antisense molecules, designed to target the desired cells (e.g., antisense molecule linked to peptides or antibodies that specifically bind receptors or antigens expressed on the target cell surface) can be administered systemically.

The invention also includes the use of a recombinant DNA construct in which the antisense oligonucleotide or polynucleotide is placed under the control of a promoter. The use of such a construct to transfect target cells in the patient results in the transcription of single stranded RNAs that form complementary base pairs with the endogenous LIF transcripts and thereby prevent translation of the LIF mRNA. Such a vector can remain episomal or become chromosomally integrated, as long as it is transcribed to produce the antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any appropriate promoter. Such promoters can be inducible or constitutive. Any appropriate methods for gene therapy available in the art can be used.

Ribozymes can also be used in accordance with the invention as LIF antagonists (for a review see, for example, Rossi, Current Biology 4:469-471 (1994)). The mechanism of ribozyme action involves sequence specific hybridization of the ribozyme molecule to complementary target RNA, followed by a endonucleolytic cleavage. Ribozyme molecules suitable for use in the invention include one or more sequences complementary to the LIF mRNA and the catalytic sequence responsible for mRNA cleavage (see U.S. Pat. No. 5,093,246). Ribozyme molecules designed to catalytically cleave LIF mRNA transcripts can be used to prevent translation of LIF mRNA. While ribozymes that cleave mRNA at site specific recognition sequences can be used to destroy LIF mRNAs, the use of hammerhead ribozymes is preferred. Hammerhead ribozymes cleave mRNAs at locations dictated by flanking regions that form complementary base pairs with the target mRNA. The construction and production of hammerhead ribozymes is well known in the art (see, for example, Haseloff and Gerlach, Nature 334:585-591 (1988)).

As in the antisense approach, the ribozymes can be composed of modified oligonucleotides (e.g., for improved stability, targeting, etc.). A preferred method of delivery involves using a DNA construct “encoding” the ribozyme under the control of a strong constitutive pol III or pol II promoter, so that transfected cells will produce sufficient quantities of the ribozyme to destroy endogenous LIF messages and inhibit translation.

RNA interference can also be used to effect inhibition of LIF expression (see, for example, Lagos-Quintana et al, Science 294:853-858 (2001); Lau et al, Science 294:858-862 (2001); Lee and Ambros, Science 294:862-864 (2001); Sharp, Genes Dev. 15:485-490 (2001); Elbashir et al, Nature 411:494-498 (2001); Fire et al, Nature 391:806-811 (1998); Hammond et al, Nature 404:293-295 (2000); Hunter, Curr. Biol. 10:R137-40 (2000); Bosher et al Nat. Cell. Biol. 2:31-36 (2000); and Zamore et al, Cell 101:25-33 (2000)).

An effective amount of antagonist(s) to be employed therapeutically will depend, for example, upon the nature of the antagonist, the route of administration, and the condition of the patient. One skilled in the art can readily establish an optimum dosing regimen.

LIF antagonists can be administered systemically, orally, or in the subcutaneous tissue, intramuscularly, intranasally or intravenously. Targeted delivery can be effected using thymus-specific ligands/chemokines. Antagonists can also be administered locally, directly to the thymus, to minimize systemic side effects. Direct administration to the thymus, which can be accomplished using, for example, a mediastinoscope, is preferred when the antagonist is an antisense construct or ribozyme.

The approaches described above can be used alone or as one component of a combination therapy that includes, for example, the administration of agents (e.g., cytokines and/or hormones) that promote thymic activation and/or growth.

While the therapeutic approaches described herein are suitable for use in humans, it will be appreciated that they have applicability in non-human animals (e.g., non-human mammals) as well.

In another embodiment, the present invention relates to methods of screening test compounds for their ability to inhibit LIF induction of thymic corticosteriods and resulting thymic atrophy. One such method involves injecting a rodent (e.g., a mouse) with LPS in an amount sufficient to induce thymic atrophy. The LPS-treated animal can be pretreated or treated (e.g., at various time intervals) with the test compound and the effect of the test compound on the LPS-induced thymic atrophy (and/or systemic and/or intrathymic LIF and/or corticosteroid levels) determined by comparison with control-treated animals. One example of such an method is given in Example 2.

Certain aspects of the invention can be described in greater detail in the non-limiting Examples that follows. (See also U.S. application Ser. Nos. 09/550,099 and 09/973,959 and LIF antagonists described in U.S. Pat. Nos. 5,837,241 and 6,387,875.)

### EXAMPLE 1

Experimental Details

Mice

Normal and adrenalectomized female BALB/c mice (6-8 week) were purchased from The Jackson Laboratory (Bar Harbor, Me.). Animals were housed in specific pathogen free conditions in the Duke University Vivarium Barrier (Durham, N.C.). Mouse handling and experimental procedures were conducted in accordance with AALAC and the Duke University IACUC guidelines for animal care and use.

Treatment of Mice and Sample Preparation

Animals were treated with either E. coli LPS (Sigma, L2880, St. Louis, Mo.), recombinant murine LIF (R&D Systems, Minneapolis, Minn.), goat IgG (Sigma) or anti-mouse LIF polyclonal goat IgG (R&D Systems) and metyrapone (Sigma). Mouse thymus tissue was excised following euthanasia. Half of each thymus was snap frozen in a dry ice/ETOH bath and stored in liquid nitrogen for RNA extraction and the other half was teased into a single-cell suspension as previously described (Sempowski et al, Journal of Immunology 164:2180 (2000)).

Murine LIF ELISA

Serum levels of mouse LIF were determined by ELISA. Ninety-six-well ELISA plates (Becton Dickinson) were coated overnight at 4° C. with polyclonal goat anti-mouse LIF (R&D Systems) at 2 μg/ml in 0.1 M NaHCO₃. Plates were washed three times with Wash buffer [(1× PBS with 0.05% Tween 20 (v/v)] and blocked at 37° C. for 2 hours with Block buffer (1× PBS, 3% BSA, 0.1% azide, and 5% FBS). Plates were then washed and a dilution series of either mouse LIF (0-5,000 pg/ml; R&D Systems) or mouse sera was added to the plates and incubated for 2 hours at 37° C. Plates were next incubated with a 1:500 dilution of rat anti-mouse LIF polyclonal sera for 2 hours at 37° C. Plates were washed and reacted with anti-rat Ig-horseradish peroxidase (Southern Biotechnology Associates, Inc., Birmingham, Ala.) for 30 minutes at room temperature. Plates were washed, developed with TMB (Kirkegaard and Perry, Gaithersburg, Maryland) and absorbance was read (450 nm). The concentration of LIF in samples was determined using the standard curve of mouse LIF.

Corticosterone Assay

Plasma corticosterone levels in mice were determined by RIA (ICN, Costa Mesa, Calif.) according to the manufacturer's protocol. All animals were bleed under anesthesia between 9 am and 11 am.

RNA Isolation and RNase Protection Assays

Steady-state levels of murine LIF mRNA in thymus were determined using the multi-probe RiboQuant RNase Protection System (Pharmingen, San Diego, Calif.) as previously described (Sempowski et al, Journal of Immunology 164:2180 (2000)). Ten micrograms of total RNA was hybridized overnight with a ³²P-labeled riboprobe (mCK4, Pharmingen) and samples processed as described in the manufacturer's instructions.

Immunophenotyping and Flow Cytometry

Phenotypic analysis of thymocyte suspensions was performed on a FACSVantage SE (Becton Dickinson) using fluorescein isothiocyanate-CD3, phycoerythrin-CD4, and phycoerythrin cychrome 5-CD8 (Pharmingen) as previously described (Sempowski et al, Journal of Immunology 164:2180 (2000)). Annexin V-FITC/propidium iodide (PI) staining was used to determine thymocyte apoptosis, necrosis and viability (Coulter).

Murine Fetal Thymic Organ Culture (FTOC)

FTOC were established following the methods described by Vacchio et al. (Journal of Experimental Medicine 185:2033 (1997)). Briefly, thymic lobes were removed from 17 day gestation fetal mice and were cultured in 6-well plates on Gelfoam (Pharmacia & Upjohn, Kalamazoo, Mich.) rafts. Cultures (2-4 lobes/well) were treated with murine LIF (R&D Systems) with or without metyrapone and then harvested by disruption against a 70 μm cell strainer (Becton Dickinson) with a 1 cc plunger (Becton Dickinson). Total thymocyte number per lobe was determined by hemacytometer count with trypan dye exclusion.

Statistical Analysis

Student's t test was used to compare the means for two data sets. Differences were considered significantly different with P<0.05.

Results

LIF is a Mediator of LPS-Induced Acute Thymic Atrophy

Intraperitoneal injection of gram-negative bacteria (E. coli) or E. coli-derived LPS into C57BL/6 mice has been reported to induce thymic atrophy. Thymus weight, cellularity and thymocyte viability and absolute number of CD4/CD8 double positive (DP) thymocytes decreased at 3 hours and reached the lowest level at 24-72 hours (Wang et al, Journal of Immunology 152:5014 (1994), Zhang et al, Infection & Immunity 61:5044 (1993)).

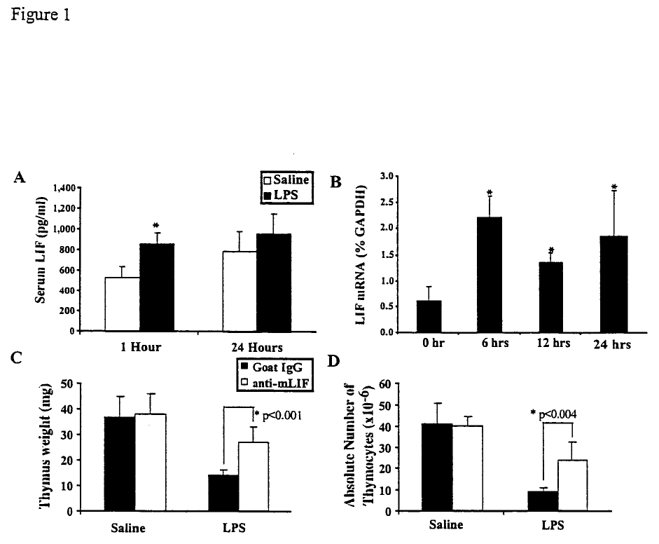

Serum from LPS-treated animals was assayed for the presence of LIF protein by ELISA to determine if LPS induced systemic LIF. LPS induced a significant rise in serum LIF 1 hour after LPS injection (P<0.05) (FIG. 1A). Thymus LIF mRNA levels were next determined in animals treated with LPS (100 μg IP for 0, 6, 12 and 24 hours) (FIG. 1B). Significant induction of intrathymic LIF mRNA was found at 6 hours that persisted for 24 hours (P<0.05). Taken together, these data demonstrated that systemic LPS treatment induced both systemic and intrathymic LIF.

It was next questioned whether anti-LIF polyclonal antibody could inhibit LPS-induced acute thymic atrophy. BALB/c mice were pre-treated with either non-immune purified goat IgG (1 mg) or purified goat anti-LIF IgG (1 mg) 6 hours before receiving either 100 μg LPS or saline IP. Thymus tissue was studied 24 hours after LPS treatment (FIGS. 1C, D). It was found that treatment with purified goat anti-mLIF polyclonal antibody inhibited LPS-induced acute thymic atrophy by 52% (P<0.001). Thus, LIF is a key in vivo mediator of LPS-induced acute thymic atrophy.

LIF Mediates Thymic Atrophy Indirectly by Induction of Corticosteroids

It has been previously shown that injection of LIF into mice induces acute thymic atrophy (Sempowski et al, Journal of Immunology 164:2180 (2000); Metcalf et al, Blood 76:50 (1990)). To determine if LIF directly induces apoptosis in thymocytes in vitro, freshly isolated thymocytes were cultured for 7 days with medium alone or 10 ng/ml LIF. Using Annexin V/PI staining, no significant effect of LIF treatment on thymocyte apoptosis/viability was observed. These data suggested that LIF mediates thymic atrophy via an indirect mechanism.

Melmed and colleagues have demonstrated the roles the IL-6 family of cytokines (LIF, Oncostatin M, IL-6) in regulating pituitary ACTH production. They have also shown that LPS can induce LIF and LIF receptor upregulation in the hypothalamus and pituitary, and suggested that LPS may act on the pituitary through LIF to induce a rise in systemic corticosteroid levels (Woo et al, AIDS Research & Human Retroviruses 15:1377 (1999)).

Corticosteroids have been implicated in mediating thymocyte death in sepsis animal models. All lymphocytes express functional corticosteroid receptors (Plaut, Annual Review of Immunology 5:621 (1987)), and murine and human immature thymocytes are sensitive to corticosteroid-induced apoptosis (Wylie, Nature 284:555 (1980)). Recently, Vacchio et al. have demonstrated intrathymic production of corticosteroids by thymic stromal cells (Vacchio et al, Journal of Experimental Medicine 179:1835 (1994)). They have also shown that physiologic corticosteroid levels (10⁻⁹ M) are required for maturation of immature thymocytes and for normal positive and negative thymocyte selection. In contrast, stress and pharmacologic levels (10⁻⁸ M, 10⁻⁷ M) of corticosteroids in the thymus induce thymocyte apoptosis and acute thymic atrophy Wylie, Nature 284:555 (1980)). It was found that LIF treatment of mice induced a rapid elevation in plasma corticosterone that peaked 1 hr after 3 μg IP LIF injection (258±14 ng/ml vs saline treated 178±9 ng/ml).

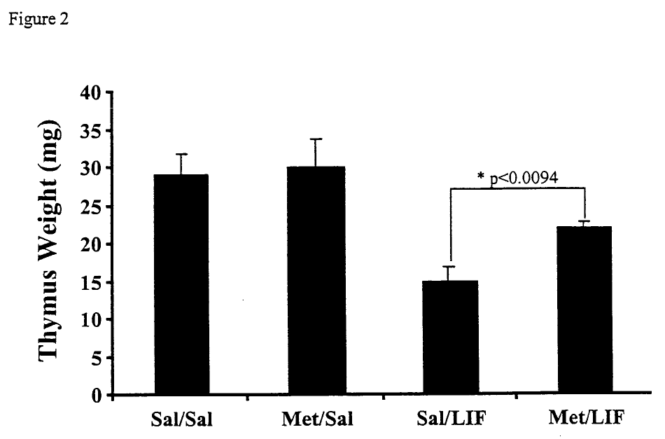

To determine if corticosteroid production was required for LIF-induced acute thymic atrophy, mice were pretreated (IP) for 24 hours with metyrapone, an inhibitor of the p450C 11-β-hydroxylase steroidogenic enzyme (FIG. 2). Metyrapone inhibited LIF-induced acute thymic atrophy by 43% (P<0.0094). Metyrapone can affect both adrenal and intrathymic corticosteroid production. To determine if LIF acted on the thymus or on the hypothalamic-pituitary adrenal axis, in vivo studies were next performed using adrenalectomized mice and in vitro studies were performed using isolated fetal thymic organ cultures (FTOCs).

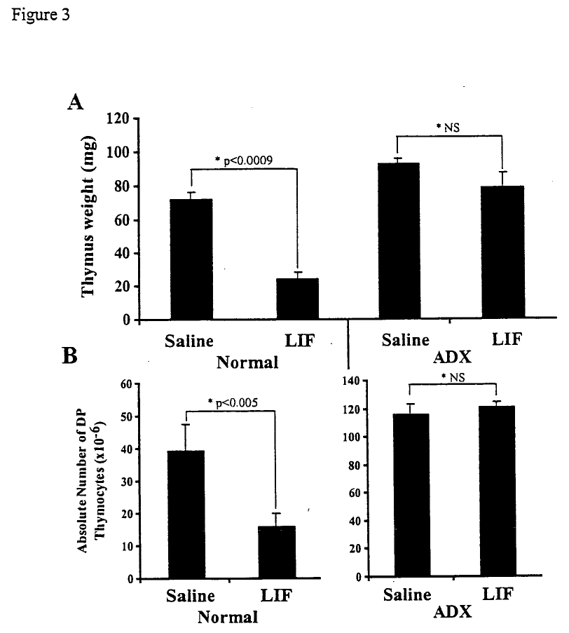

Normal and bilaterally adrenalectomized BALB/c mice were treated with LIF (3 μg, 3× a day) or saline for 3 days, and the degree of thymic atrophy assessed. As previously seen, thymus weight (FIG. 3A) and CD4/CD8 DP thymocytes (FIG. 3B) were significantly reduced in LIF-treated normal mice (P<0.05). However, LIF-treated adrenalectomized animals had thymus weight and number of CD4/CD8 DP thymocytes that were not significantly different from saline injected animals (FIG. 3). These data demonstrated that systemic LIF induction of thymic atrophy requires adrenal production of corticosteroids. However, if LIF-induced intrathymic corticosteroid production was also playing a role in mediation of thymic atrophy, the lack of thymocyte depletion by LIF in adrenalectomized animals in these experiments was perplexing. Either intrathymic LIF-induced corticosteroid production did not play a role in LPS/LIF induced thymic atrophy, or systemic LIF treatment does not induce LIF in the thymus, or does not reach thymic levels sufficient to induce apoptosis. To evaluate the status of LIF mRNA in the thymus of LIF injected mice, thymus LIF mRNA levels were determined in control and LIF-treated mice. Unlike LPS-treated mice, no induction of thymic LIF mRNA by systemic LIF administration was found. Thus, it is possible that the effect of systemic LIF was only via its effect on adrenal corticosteroid production.

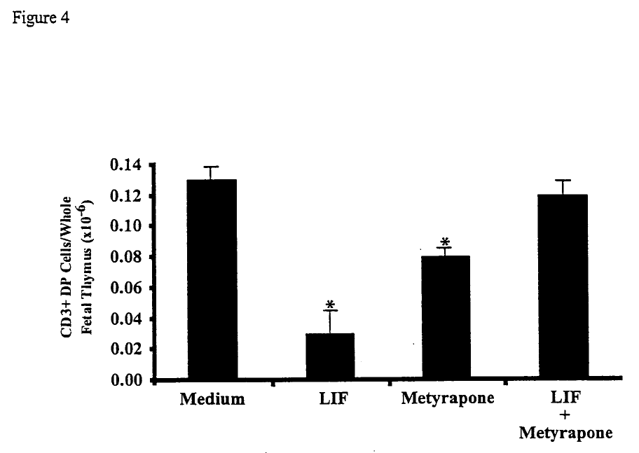

To directly determine if LIF can induce intrathymic corticosteroid production and thymocyte depletion, 17 day fetal thymic lobes in FTOC were treated with LIF (10 ng/ml) in the presence or absence of metyrapone (FIG. 4). Absolute numbers of CD3⁺, CD4/CD8 DP thymocytes were quantitated as a measure of in vitro LIF-induced thymic atrophy after 72 hours. LIF-induced significant depletion of DP thymocytes (77%, P<0.05), and metyrapone alone induced a slight decrease in DP thymocytes (8%, P<0.05) (FIG. 4). However, the addition of metyrapone to LIF-treated FTOC prevented LIF-induce depletion of DP thymocytes. These data demonstrated that elevated intrathymic LIF levels can induce thymocyte depletion via induction of thymic corticosteroids.

### EXAMPLE 2

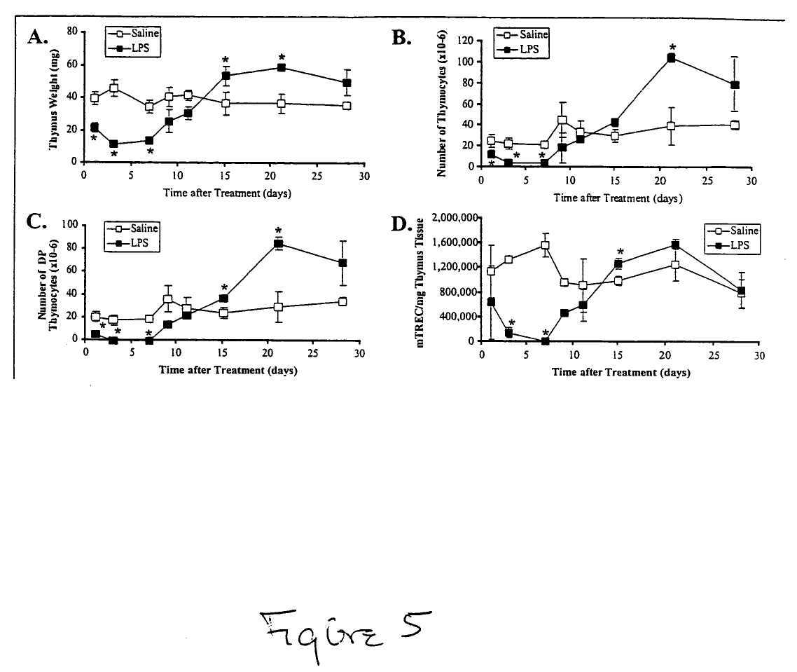

Model of LPS-induced acute thymic atrophy and thymus regeneration: Intraperitoneal injection of gram-negative bacteria (E. coli) into B6 mice has been reported to induce thymic atrophy in mice (Wang et al, Journal of Immunology 152:5014 (1994)). Thymus weight, cellularity and thymocyte viability began decreasing at 3 hours and reached the lowest level at 72 hours. In this study, purified gram-negative bacterial lipopolysaccharide (LPS) was used to define a model of acute thymic atrophy and regeneration. BALB/c female mice were injected with a sub-lethal dose of LPS (100 μg IP) and thymus weight, cellularity, thymocyte phenotype (CD4/CD8) and mTREC levels were monitored (Sempowski et al, Mol. Immunol. 38(11):841-8 (2002)) (FIG. 5). During the first seven days after LPS injection, a significant drop in thymus weight and absolute number of thymocytes (p<0.05) were observed (FIGS. 5A, 5B). Flow cytometry analysis of thymocyte subset phenotype revealed a significant decline in the frequency and absolute number of CD5/CD8 DP cortical thymocytes (FIG. 5C). A quantitative assessment of thymopoiesis using the mTREC assay confirmed a loss of thymopoiesis following LPS challenge (FIG. 5D). From day 7 through 28, a rebound of thymopoiesis was observed in the LPS-treated animals. The thymus weight, cellularity, phenotype and mTREC levels returned to levels similar to saline-treated animals. A striking result from this study was that rebound of the thymus continued beyond that of saline controls, with augmented thymus growth from day 15 through day 28 (FIGS. 5A-5D). Regeneration of the thymus in this model was characterized by increased overall weight of the thymus, cellularity, number of cortical DP thymocytes and increased frequency of cells rearranging their TCRA locus (mTRECS normalized per mg tissue). Using a 1 hour pulse with bromo-deoxyuridine (BrdU; 1 mg IP) and CD4/CD8 surface phenotype with intracellular staining for BrdU incorporation, the number of cells proliferating for each thymocyte subset was quantitated. With this assay, it has been possible to demonstrate that the number of proliferating DP thymocytes in rebounding thymus tissue from LPS-treated animals significantly exceeded that from saline-treated animals (p<0.05).

Test for experimental therapeutics: Animals will be given a sublethal dose of LPS (100 μg IP) that induces acute thymic atrophy within 24 hours and pretreated or treated (e.g., IP, subcutaneously or IV) at various time intervals with a cytokine, antibody, hormone, and/or other experimental therapeutic inhibitory agent (when the experimental therapeutic is a protein, it will be appreciated that the protein can be administered directly or a construct comprising a nucleic acid encoding the protein can be administered (e.g., the encoding sequence can be present in a viral vector)). All experiments will include unmanipulated animals, saline-treated (no LPS) animals, and appropriate control-treated animals for the various treatments (e.g., species specific non-immune Ig as a control for anti-LIF polyclonal antibody). A minimum of three animals per each group and usually six animals per group will be studied, with appropriate dose of each experimental treatment determined in pilot studies.

All documents cited above are hereby incorporated in their entirety by reference.

What is claimed is:

1. A method of treating or preventing gram-negative endotoxin-induced thymic atrophy comprising administering to a patient in need of said treatment or prevention an amount of an agent that inhibits leukemia inhibitory factor (LIF) induction of thymic corticosteriods sufficient to effect said treatment or prevention.

2. The method according to claim 1 wherein said agent is a LIF antagonist.

3. The method according to claim 2 wherein said agent inhibits intrathymic production or function of LIF.

4. The method according to claim 2 wherein said agent inhibits intracellular or membrane associated events that occur between LIF and a LIF receptor.

5. The method according to claim 2 wherein said agent is an antibody, or fragment thereof, that inhibits interaction between LIF and a LIF receptor.

6. The method according to claim 2 wherein said agent is a soluble LIF receptor, or mimic thereof, that inhibits interaction between LIF and a LIF receptor.

7. The method according to claim 1 wherein said agent inhibits LIF expression.

8. The method according to claim 7 wherein said agent is an antisense molecule or a ribozyme.

9. The method according to claim 7 wherein RNA interference is used to effect said inhibition of LIF expression.

10. The method according to claim 1 wherein said antagonist is administered directly to the thymus.

11. The method according to claim 1 wherein said method further comprises administering to said patient a compound that promotes thymic activation or growth.

12. The method according to claim 1 wherein said patient is a human.

13. A composition comprising a LIF antagonist and a compound that promotes thymic activation or growth.

14. A method of screening a test compound for the ability to inhibit LIF induction of thymic atrophy comprising:
i) administering LPS to a rodent in an amount sufficient to induce thymic atrophy,
ii) treating said rodent with a test compound,
iii) determining the effect of said test compound on systemic or intrathymic corticosteroid levels, as compared to an LPS-treated control rodent,
wherein a test compound that inhibits LPS-induced corticosteroid production inhibits said LIF induction of thymic atrophy.

15. The method according to claim 14 wherein said rodent is a mouse.
